(19) United States
(12) Patent Application Publication
(10) Pub. No.: US 2025/0248755 A1
(43) Pub. Date: Aug. 7, 2025

(54) DRIVE MECHANISMS FOR SURGICAL INSTRUMENTS

(71) Applicant: Covidien LP, Mansfield, MA (US)

(72) Inventor: Sean W. Miller, Broomfield, CO (US)

(73) Assignee: Covidien LP, Mansfield, MA (US)

(21) Appl. No.: 19/014,666

(22) Filed: Jan. 9, 2025

Related U.S. Application Data
(60) Provisional application No. 63/550,842, filed on Feb. 7, 2024.

(51) Int. Cl. A61B 18/14; A61B 18/00

(52) U.S. Cl. CPC A61B 18/1445; A61B 2018/00172; A61B 2018/00184; A61B 2018/0091

(57) ABSTRACT

A surgical instrument includes a housing, a distally spaced end effector assembly including first and second jaw members, an elongate drive coupled to either or both jaw members, a movable handle, and a drive mechanism. The drive mechanism includes a first four-bar mechanical linkage assembly wherein the movable handle defines or is coupled to a handle linkage of the first four-bar mechanical linkage assembly; and a second four-bar mechanical linkage assembly wherein a drive linkage of the second four-bar mechanical linkage assembly is coupled to the elongate drive such that movement of the drive linkage translates the elongate drive. The first and second four-bar mechanical linkage assemblies are coupled to one another, thereby coupling the movable handle with the elongate drive such that movement of the movable handle moves either or both of the jaw members.

### CROSS-REFERENCE TO RELATED APPLICATIONS

This application claims priority to U.S. Patent Application No. 63/550,842, filed Feb. 7, 2024, which applications are incorporated herein by reference in their entireties.

### FIELD

The present disclosure relates to surgical instruments and, more particularly, to drive mechanisms for surgical instruments such as, for example, to actuate end effector assemblies of the surgical instruments.

### BACKGROUND

A surgical forceps is a pliers-like surgical instrument that relies on mechanical action between its jaw members to grasp, clamp, and constrict tissue. Energy-based surgical forceps utilize both mechanical clamping action and energy, e.g., monopolar Radio Frequency (RF), bipolar RF, microwave, ultrasonic, light, thermal, combinations thereof, and/or other suitable energy, to heat tissue to thereby treat, e.g., seal and/or cut, tissue grasped between jaw members of the energy-based surgical forceps.

### SUMMARY

As used herein, the term “distal” refers to the portion that is being described which is farther from an operator, while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with the present disclosure is a surgical instrument including a housing, an end effector assembly, an elongate drive, a movable handle, and a drive mechanism. The end effector assembly is distally spaced from the housing and includes first and second jaw members. At least one of the first or second jaw members is movable relative to the other between a spaced apart position and an approximated position. The elongate drive extends from the housing to the end effector assembly and is coupled to the at least one of the first or second jaw members such that translation of the elongate drive moves the at least one of the first or second jaw members relative to the other. The movable handle is coupled to the housing and movable relative to the housing between an un-actuated position and an actuated position. The drive mechanism is at least partially disposed within the housing and includes a first four-bar mechanical linkage assembly and a second four-bar mechanical linkage assembly. The movable handle defines or is coupled to a handle linkage of the first four-bar mechanical linkage assembly. A drive linkage of the second four-bar mechanical linkage assembly is coupled to the elongate drive such that movement of the drive linkage of the second four-bar mechanical linkage assembly translates the elongate drive. The first and second four-bar mechanical linkage assemblies are coupled to one another, thereby coupling the movable handle with the elongate drive such that movement of the movable handle from the un-actuated position towards the actuated position translates the elongate drive to thereby move the at least one of the first or second jaw members from the spaced-apart position towards the approximated position.

In an aspect of the present disclosure, the first and second four-bar mechanical linkage assemblies are configured such that movement of the movable handle in a rotational direction drives rotation of the drive linkage of the second four-bar mechanical linkage assembly in the same rotational direction. In the same or other aspects, the first and second four-bar mechanical linkage assemblies are configured such that movement of the movable handle in a direction translates the elongate drive in the same direction. In alternative aspects, the first and second four-bar mechanical linkage assemblies may be configured such that movement of the movable handle in a direction translates the elongate drive in an opposite direction.

In another aspect of the present disclosure, the first and second four-bar mechanical linkage assemblies share a common pivot. The common pivot couples the first and second four-bar mechanical linkage assemblies with one another. The common pivot may be fixed relative to the housing.

In another aspect of the present disclosure, a linkage of each of the first and second four-bar mechanical linkage assemblies is pivotably coupled to the common pivot. These linkages, in aspects, may be disposed in fixed orientation relative to one another.

In yet another aspect of the present disclosure, the first four-bar mechanical linkage assembly includes a fixed linkage and three movable linkages. The handle linkage defines one of the three movable linkages.

In still another aspect of the present disclosure, the housing defines the fixed linkage of the first four-bar mechanical linkage assembly.

In another aspect of the present disclosure, the second four-bar mechanical linkage assembly includes a fixed linkage and three movable linkages. The drive linkage defines one of the three movable linkages of the second four-bar mechanical linkage assembly.

In still yet another aspect of the present disclosure, the housing defines the fixed linkage of the second four-bar mechanical linkage assembly.

In another aspect of the present disclosure, the movable handle is pivotably coupled to the housing about a fixed pivot of the first four-bar mechanical linkage assembly. Alternatively or additionally, the drive linkage is pivotably coupled to the housing about a fixed pivot of the second four-bar mechanical linkage assembly.

In yet another aspect of the present disclosure, the drive mechanism further includes a mandrel coupled to the elongate drive. The drive linkage is coupled to the mandrel such that movement of the drive linkage of the second four-bar mechanical linkage assembly translates the mandrel to thereby translate the elongate drive.

In still another aspect of the present disclosure, the mandrel is coupled to the elongate drive by a compression spring of the drive mechanism such that translation of the mandrel applies force to the compression spring which, in turn, applies force to the elongate drive.

In still yet another aspect of the present disclosure, the movable handle defines the handle linkage of the first four-bar mechanical linkage assembly.

Another surgical instrument provided in accordance with the present disclosure includes a housing, an end effector assembly, an elongate drive, a movable handle, and a drive mechanism. The end effector assembly is distally spaced from the housing and includes first and second jaw members. At least one of the first or second jaw members is movable relative to the other between a spaced apart position and an approximated position for grasping tissue therebetween. The elongate drive extends from the housing to the end effector assembly and is coupled to the at least one of the first or second jaw members such that translation of the elongate drive moves the at least one of the first or second jaw members relative to the other. The movable handle is coupled to the housing and movable relative to the housing between an un-actuated position and an actuated position. The drive mechanism is at least partially disposed within the housing and includes a plurality of movable linkages coupled between the movable handle and the elongate drive such that movement of the movable handle from the un-actuated position towards the actuated position translates the elongate drive to thereby move the at least one of the first or second jaw members from the spaced-apart position towards the approximated position. The plurality of movable linkages includes a handle linkage pivotably coupled to the housing about a first fixed pivot, a drive linkage pivotably coupled to the housing about a second fixed pivot, a first intermediate linkage coupled to the handle linkage, a second intermediate linkage coupled to the drive linkage, and first and second angled linkages coupled between the first and second intermediate linkages, the first and second angled linkages pivotably coupled to the housing about a third fixed pivot and defining a fixed angle therebetween.

In an aspect of the present disclosure, the plurality of movable linkages is configured such that movement of the movable handle in a rotational direction drives rotation of the drive linkage in the same rotational direction. In such or other aspects, the plurality of movable linkages are configured such that movement of the movable handle in a direction translates the elongate drive in the same direction. In alternative aspects, the plurality of movable linkages may be configured such that such that movement of the movable handle in a rotational direction drives rotation of the drive linkage in the opposite rotational direction and/or such that movement of the movable handle in a direction translates the elongate drive in the opposite direction.

In another aspect of the present disclosure, the handle linkage, the first intermediate linkage, the first angled linkage, and the housing define a first four-bar mechanical linkage assembly. Additionally or alternatively, the drive linkage, the second intermediate linkage, the second angled linkage, and the housing define a second four-bar mechanical linkage assembly.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a side view of an electrosurgical system in accordance with the present disclosure including an electrosurgical forceps and an electrosurgical generator;

FIG. 2 is an enlarged, perspective view of an end effector assembly of the electrosurgical forceps of the system of FIG. 1 with jaw members of the end effector assembly disposed in a spaced apart position;

FIG. 3 is a side view of the end effector assembly of FIG. 2 with the jaw members of the end effector assembly disposed in an approximated position;

FIG. 4 is a side view of a proximal portion of the electrosurgical forceps of FIG. 1 with portions removed to illustrate a drive mechanism of the electrosurgical forceps of FIG. 1 coupling a movable handle with an elongate drive, wherein the movable handle is disposed in an un-actuated position corresponding to the spaced apart position of the jaw members;

FIG. 5 is a side view of the proximal portion of the electrosurgical forceps of FIG. 1 with portions removed to illustrate the drive mechanism, wherein the movable handle is disposed in an actuated position corresponding to the approximated position of the jaw members;

FIG. 6 is a free body diagram of the drive mechanism of FIG. 4 corresponding to the un-actuated position of the movable handle;

FIG. 7 is a free body diagram of the drive mechanism of FIG. 4 corresponding to the actuated position of the movable handle;

FIG. 8 is a free body diagram of another drive mechanism configured for use with the electrosurgical forceps of FIG. 1, corresponding to the un-actuated position of the movable handle; and

FIG. 9 is a free body diagram of the drive mechanism of FIG. 8, corresponding to the actuated position of the movable handle.

### DETAILED DESCRIPTION

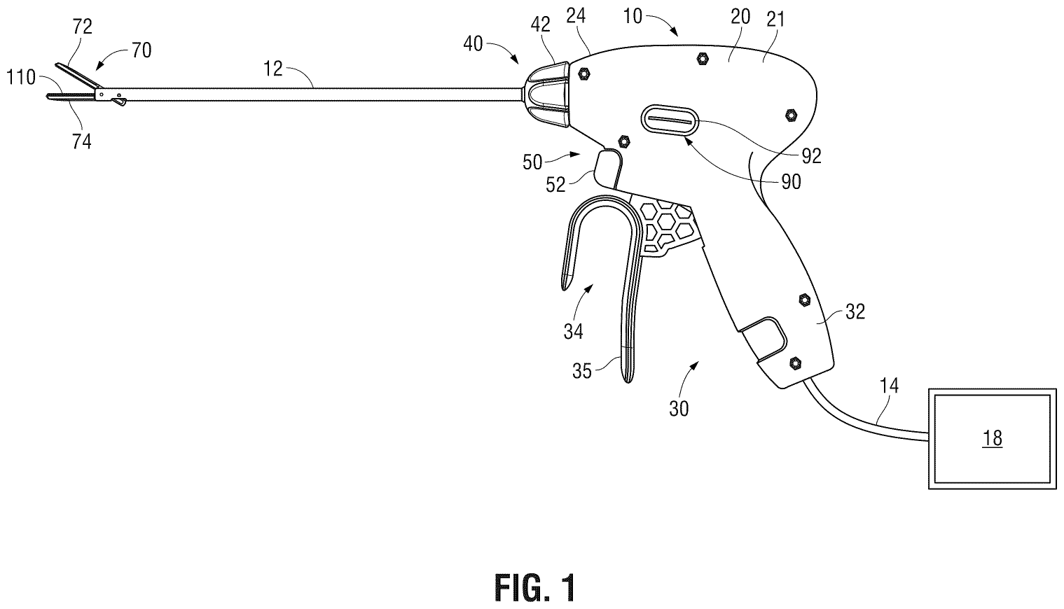

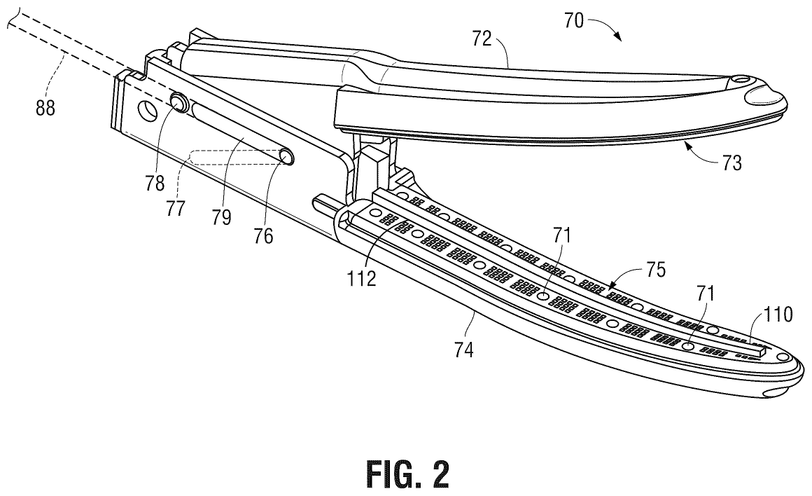

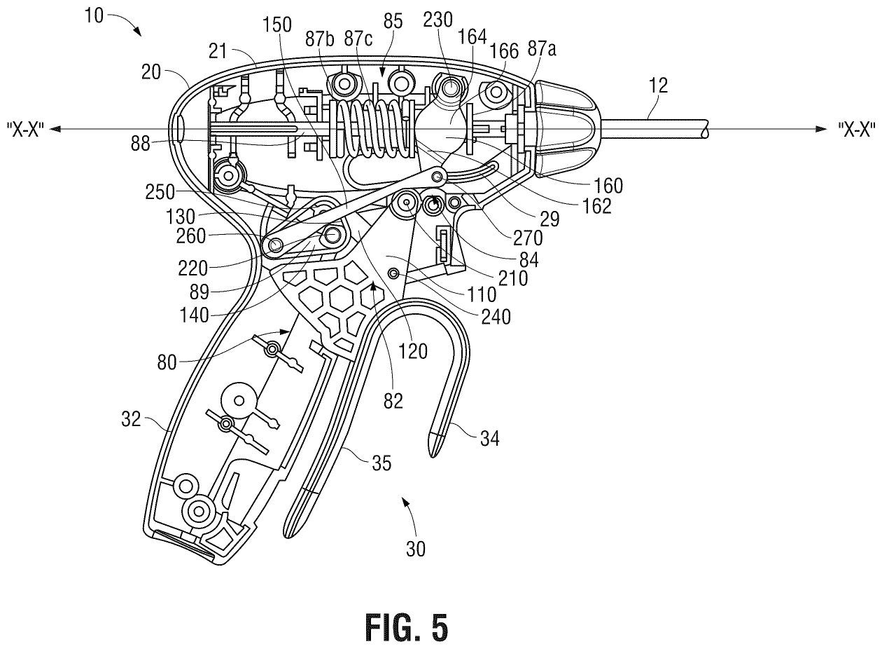

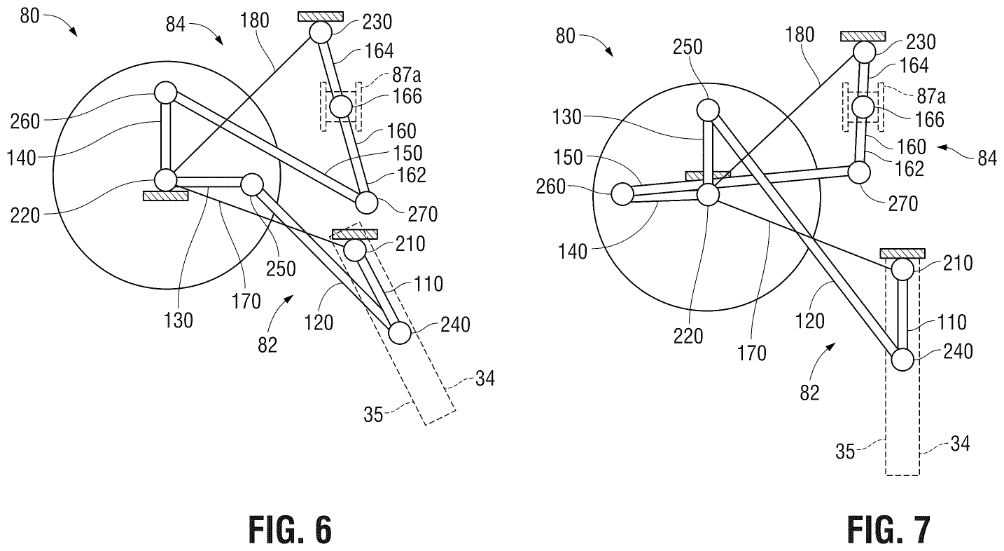

Referring to FIGS. 1-3, an electrosurgical system 2 provided in accordance with the present disclosure includes an electrosurgical forceps 10 and an electrosurgical generator 18. Forceps 10 includes a housing 20, a handle assembly 30, a rotating assembly 40, first and second activation assemblies 50, 90, an end effector assembly 70, a drive mechanism 80 (FIGS. 5 and 6), and an elongate drive 88. End effector assembly 70 includes first and second jaw members 72, 74, at least one of which is movable relative to the other (e.g., about pivot pin 78 (FIG. 2) or other suitable structure(s)) between a spaced apart position and an approximated position to grasp tissue (e.g., to enable sealing and/or dividing of the grasped tissue). End effector assembly 70 further includes an energy-based cutting element 110 such as, for example, a cutting electrode, configured to supply energy to tissue to cut tissue grasped between jaw members 72, 74 or otherwise in contact with cutting element 110. As an alternative to a cutting electrode 110, end effector assembly 70 may include a mechanical knife (not shown) configured for translation between jaw members 72, 74 to cut tissue grasped therebetween.

Forceps 10 further includes an outer shaft 12 that has a proximal end portion operably engaged to a body 21 of housing 20 and a distal end portion operably engaged to end effector assembly 70. Forceps 10 also includes an electrosurgical cable 14 configured to connect forceps 10 to generator 18 to enable generator 18 to communicate with forceps 10 and control the supply of electrosurgical energy to end effector assembly 70 of forceps 10 for sealing tissue grasped between first and second jaw members 72, 74 and for cutting tissue disposed between first and second jaw members 72, 74 or otherwise in contact with cutting element 110 of end effector assembly 70.

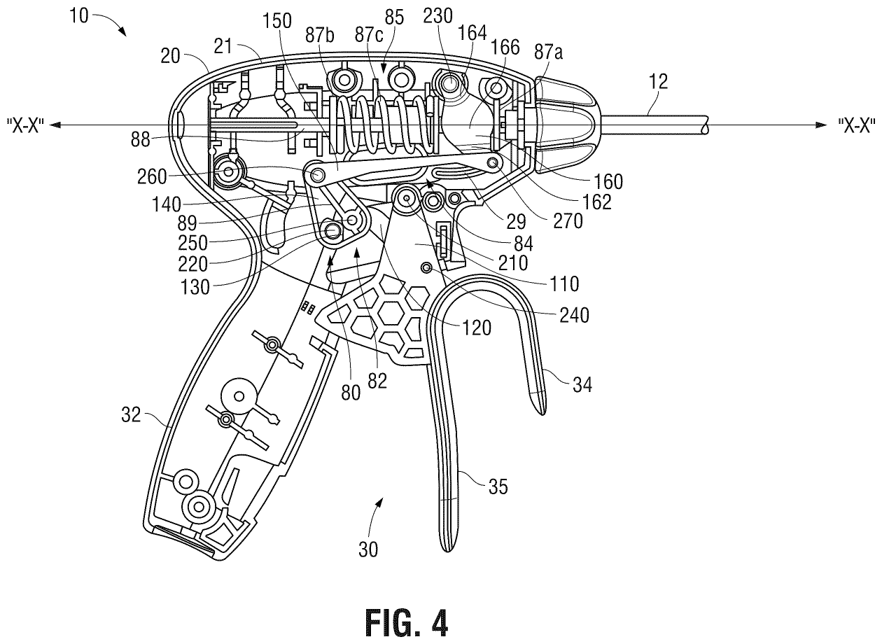

Continuing with reference to FIGS. 1-3, handle assembly 30 includes a fixed handle 32 and a movable handle 34. Fixed handle 32 is integrally associated with housing 20, depending from body 21 thereof, and movable handle 34 is movable relative to fixed handle 32 to actuate drive mechanism 80 (FIGS. 4 and 5), as detailed below. Actuation of drive mechanism 80 (FIGS. 4 and 5), in turn, translates elongate drive 88 through outer shaft 12 and relative to end effector assembly 70 to move either or both of jaw members 72, 74 between the spaced apart and approximated positions.

Elongate drive 88, more specifically, is configured to translate through outer shaft 12 to drive movement of cam pin 76 through cam slots 77, 79 defined within respective first and second jaw members 72, 74 to urge jaw member 72 to pivot about pivot pin 78 and relative to jaw member 74 from the spaced apart position to the approximated position to grasp tissue between first and second jaw members 72, 74 and apply a closure force to the grasped tissue. Elongate drive 88 may be formed from one or more drive elements such as, for example and without limitation, one or more shafts, tubes, cables, plates, linkages, etc. In aspects, one or more portions of elongate drive 88 may be configured to rotate, articulate, bend, slide, or otherwise move relative to one or more other portions (of the same drive element and/or of one or more different drive elements) of elongate drive 88.

In order to drive relative movement of cam pin 76 through cam slots 77, 79, elongate drive 88 may be translationally fixed to cam pin 76 and outer shaft 12 translationally fixed to pivot pin 78 such that translation of elongate drive 88 moves cam pin 76 relative to jaw members 72, 74 (and, thus, cam slots 77, 79) to thereby drive relative movement of cam pin 76 through cam slots 77, 79 to pivot first jaw member 72 towards second jaw member 74 (e.g., towards the approximated position) to grasp tissue therebetween and apply the closure force to the grasped tissue. Alternatively, elongate drive 88 may be translationally fixed to pivot pin 78 and outer shaft 12 translationally fixed to cam pin 76 such that translation of elongate drive 88 moves jaw members 72, 74 (and, thus, cam slots 77, 79) relative to cam pin 76 to thereby drive relative movement of cam pin 76 through cam slots 77, 79 to pivot first jaw member 72 towards second jaw member 74 (e.g., towards the approximated position) to grasp tissue between first and second jaw members 72, 74 and apply the closure force to the grasped tissue. In configurations wherein elongate drive 88 is fixed and outer shaft 12 is movable, outer shaft 12 may thus define the elongate drive that is actuated by drive mechanism 80.

In either of the above configurations, cam slots 77, 79 may be oriented such that proximal translation of elongate drive 88 pivots first jaw member 72 towards the approximated position and distal translation of elongate drive 88 pivots first jaw member 72 towards the spaced apart position. However, the reverse is also contemplated, e.g., wherein distal translation of elongate drive 88 pivots first jaw member 72 towards the approximated position and, thus, such that proximal translation of elongate drive 88 pivots first jaw member 72 towards the spaced apart position.

Further, although end effector assembly 70 is described as a unilateral assembly, e.g., wherein second jaw member 74 is fixed relative to outer shaft 12 and first jaw member 72 is pivotable relative to second jaw member 74 and outer shaft 12, a bilateral assembly, e.g., wherein both first and second jaw members 72, 74 are pivotable relative to one another and outer shaft 12, is also contemplated. Further, instead of a cam and slot mechanism (as shown and detailed above), other suitable configurations for driving relative movement of either or both of jaw members 72, 74 are also contemplated such as, for example using one or more linkages, a lead screw, a slidable flange-aperture engagement, or any other suitable mechanism. For the purposes herein, reference to movement of jaw members 72, 74 (collectively) includes movement of only one jaw member in a unilateral configuration as well as movement of both jaw members in a bilateral configuration.

Referring still to FIGS. 1-3, each jaw member 72, 74 of end effector assembly 70 includes an electrically conductive tissue contacting surface 73, 75. Jaw members 72, 74 are configured to grasp tissue between electrically conductive tissue contacting surface 73, 75 in the approximated position thereof. Electrically conductive tissue contacting surfaces 73, 75 are adapted to connect to generator 18, e.g., via suitable electrical lead wires, electrically conductive structures, or combinations thereof extending through outer shaft 12, housing 20, and electrosurgical cable 14, to enable energization of electrically conductive tissue contacting surface 73, 75 with, for example, Radio Frequency (RF) energy at different potentials (thus defining a bipolar configuration) to enable the conduction of the RF energy between electrically conductive tissue contacting surface 73, 75 and through tissue grasped therebetween to seal the tissue. In addition to supplying energy, generator 18 is configured to monitor properties, e.g., current, voltage, power, tissue impedance, changes thereof, etc., associated with the supply of energy to implement feedback-based control of the tissue sealing process and/or to determine when tissue sealing is complete. For example, and without limitation, generator 18 may monitor tissue impedance and determine that tissue sealing is complete when a target end impedance is reached. Other suitable energy modalities, e.g., monopolar RF, ultrasonic, microwave, thermal, light, etc. are also contemplated, as are combinations thereof.

Either or both jaw member 72, 74 may further include one or more stop members 71 disposed on or otherwise associated with either or both tissue-contacting surfaces 73, 75 to maintain a minimum gap distance or gap distance within a minimum gap distance range between tissue contacting surfaces 73, 75 when jaw members 72, 74 are disposed in a fully approximated position, thus inhibiting electrical shorting. Stop member(s) 71 may be insulative, partly insulative, and/or electrically isolated from either or both tissue contacting surfaces 73, 75.

As noted above, end effector assembly 70 further includes an energy-based cutting element 110. Cutting element 110 may be configured as a cutting electrode configured to conduct RF energy to tissue or may be configured in any other suitable manner to deliver any additional or alternative form of energy, e.g., ultrasonic, thermal, light, microwave, combinations thereof, etc., to cut tissue in contact with or otherwise in close proximity to cutting element 110. In RF configurations, cutting element 110 may be energized with either or both of tissue contacting surfaces 73, 75 at different potentials to conduct energy between cutting element 110 and either or both of tissue contacting surfaces 73, 75 and through tissue disposed therebetween to cut tissue. Alternatively, cutting element 110 may be energized to conduct energy to tissue to cut tissue while a remote return electrode (not shown), e.g., a return pad, is utilized to return the energy to generator 18 to complete the electrosurgical circuit. Other electrical pathway configurations between cutting element 110 and one or more return components are also contemplated.

Cutting element 110 may be disposed within a longitudinally-extending slot 112 defined through tissue contacting surface 75 of jaw member 74 with an insulator (not shown) disposed between cutting electrode 110 and tissue contacting surface 75, thus maintaining electrical isolation between cutting element 110 and tissue contacting surface 75. Further, cutting element 110 may be positioned to oppose an insulative member (not shown) disposed on or extending through tissue contacting surface 73 of jaw member 72 in the approximated position of jaw members 72, 74 to likewise maintain electrical isolation between cutting element 110 and tissue contacting surface 73. However, other configurations are also contemplated, including configurations wherein cutting element 110 is electrically coupled to one of tissue contacting surface 73, 75.

Referring again to FIG. 1, rotating assembly 40 includes a rotation wheel 42 engaged with outer shaft 12 and rotatably disposed about a distal nose 24 of body 21 of housing 20 to enable a user to manually control the orientation of outer shaft 12 and thus, end effector assembly 70, relative to housing 20, e.g., by manipulating rotation wheel 42. In aspects, rotating assembly 40 is infinitely rotatable in either direction to similarly rotate end effector assembly 70 relative to housing 20. Alternatively, rotating assembly 40 may have a defined range of motion.

First activation assembly 50 is configured to signal generator 18 to initiate the supply of energy to tissue contacting surfaces 73, 75 of first and second jaw members 72, 74, respectively, e.g., for sealing tissue. First activation assembly 50, more specifically, includes an activation button 52 including an underlying activation switch (not shown) operably positioned such that depression of activation button 52 transitions the underlying electrical switch from a first state (e.g., an OFF state) to a second state (e.g., an ON state). The electrical switch, in turn, is adapted to electrically connect to generator 18, e.g., via one or more electrical lead wires extending from the electrical switch through housing 20 and electrosurgical cable 14 to enable communication of the state of the electrical switch to generator 18. Generator 18, more specifically, may be configured to read an output, e.g., the presence of a resistance, voltage, current, etc. and/or a value of the resistance, voltage, current, etc., established by the state or change in state of the electrical switch to thereby detect the state of the electrical switch and, thus, to detect whether the user has activated activation button 52. For example, generator 18 may read the first state of the electrical switch as corresponding to a deactivated state and the second state of the electrical switch as corresponding to an activated state. In aspects, activation button 52 is biased towards an unactivated position and, thus, the electrical switch is biased towards the deactivated state. In aspects, rather than first activation assembly 50 disposed on housing 20 and configured for activation by a finger of an operator, first activation assembly 50 may be disposed within housing 20 and configured for actuation by a movable component therein such as, for example, by a portion of movable handle 34 upon sufficient actuation of movable handle 34 relative to fixed handle 32. However, other suitable locations and/or configurations of first activation assembly 50 are also contemplated.

Second activation assembly 90 is configured to signal generator 18 to initiate the supply of energy to cutting element 110 (and, in aspects, either or both of tissue contacting surfaces 73, 75) to cut tissue grasped between jaw members 72, 74 or otherwise in contact with cutting element 110. Second activation assembly 90 includes an activation button 92 including an underlying activation switch (not shown) operably positioned such that depression of activation button 92 transitions the underlying electrical switch from a first state (e.g., an OFF state) to one or more second states (e.g., a first or low power ON state and a second or high power ON state) to thereby signal generator 18 to initiate the supply of energy to cutting element 110 (and, in aspects, either or both of tissue contacting surfaces 73, 75) for treating, e.g., cutting, tissue. Generator 18, more specifically, may be configured to read an output, e.g., the presence of a resistance, voltage, current, etc. and/or a value of the resistance, voltage, current, etc., established by the state or change in state of the electrical switch of activation assembly 90 to thereby detect the state of the electrical switch and, thus, to detect whether the user has activated activation button 92 and, where multiple modes are provided, to detect the activated mode, e.g., low power or high power. In aspects, activation button 92 is biased towards an unactivated position and, thus, the electrical switch is biased towards the deactivated state. Other suitable locations and/or configurations of second activation assembly 90 are also contemplated.

Continuing with reference to FIG. 1, generator 18 may include, for example, sensor circuitry, a controller, a high voltage power supply (“HVPS”) and first and second output stages. The HVPS provides high voltage power to the first and second output stages which convert the high voltage power into RF electrosurgical energy for delivery to tissue contacting surfaces 73, 75 (FIG. 2) and cutting element 110 of end effector assembly 70 for sealing and cutting tissue, respectively. The controller includes a processor operably connected to a non-transitory computer-readable storage medium such as a memory. The processor is operably connected to the HVPS and/or the output stages allowing the processor to control the output of generator 18, e.g., in accordance with the implemented mode(s) of operation and/or feedback data.

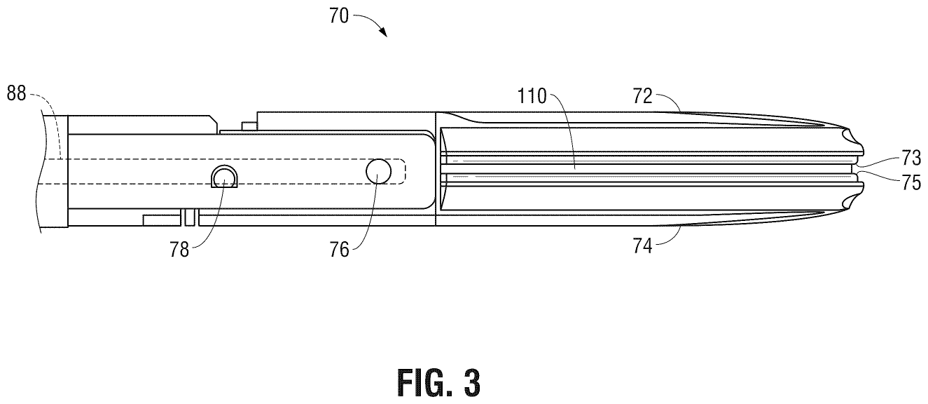

Turning to FIGS. 4 and 5, drive mechanism 80 operably couples movable handle 34 and elongate drive 88 with one another such that actuation of movable handle 34 from an unactuated position (e.g., wherein movable handle 34 is farther from fixed handle 32) towards an actuated position (e.g., wherein movable handle 34 is closer to fixed handle 32) translates elongate drive 88 through housing 20 and outer shaft 12 to move jaw members 72, 74 (FIGS. 1-3) from the spaced apart position (FIG. 2) towards the approximated position (FIG. 3). Drive mechanism 80 includes: a handle-side or first pivot and linkage assembly 82 that, in aspects, is configured as a handle-side or first four-bar mechanical linkage assembly; a drive-side or second pivot and linkage assembly 84 that, in aspects, is configured as a drive-side or second four-bar mechanical linkage assembly; and, in aspects, a force limiting assembly 85.

Force limiting assembly 85, in aspects where provided, operably couples a drive linkage 160 of drive mechanism 80 with elongate drive 88 and is configured to regulate the closure force applied to tissue grasped between jaw members 72, 74. Force limiting assembly 85 includes a mandrel 87a slidably disposed about elongate drive 88, a proximal washer 87b fixedly engaged about elongate drive 88, and a compression spring 87c. Mandrel 87a includes proximal and distal rims spaced apart from one another to define a receiving area therebetween.

Drive linkage 160 of drive mechanism 80 is operably received within the receiving area of mandrel 87a between the proximal and distal rims thereof such that, in response to actuation of movable handle 34 towards the actuated position, drive linkage 160 urges mandrel 87a proximally and such that, in response to return of movable handle 34 towards the unactuated position, drive linkage 160 urges mandrel 87a distally. Compression spring 87c is disposed about elongate drive 88 between proximal washer 87b and the proximal rim of mandrel 87a. In aspects, the proximal and/or distal ends of compression spring 87c are engaged or otherwise fixed relative to proximal washer 87b and the proximal rim of mandrel 87a, respectively. In aspects, compression spring 87c is pre-compressed in an at-rest condition of force limiting assembly 85 corresponding to the unactuated position of movable handle 34.

In use, upon initial actuation of movable handle 34 from the unactuated position towards the actuated position, drive linkage 160 is moved to urge mandrel 87a proximally. This proximal urging of mandrel 87a, in turn, urges compression spring 87c proximally into proximal washer 87b to thereby urge proximal washer 87b proximally. With proximal washer 87b fixedly engaged about elongate drive 88, as noted above, the proximal urging of proximal washer 87b translates elongate drive 88 proximally to thereby move first and second jaw members 72, 74 (FIGS. 1-3) from the spaced apart position (FIG. 2) towards the approximated position (FIG. 3).

Upon first and second jaw members 72, 74 reaching a threshold closure force applied to tissue grasped therebetween first and second jaw members 72, 74, further movement of mandrel 87a proximally against compression spring 87c, e.g., in response to further actuation of movable handle 34 towards fixed handle 32, compresses compression spring 87c rather than translating compression spring 87c proximally (due to the resistive force applied by tissue inhibiting further approximation of jaw members 72, 74) such that elongate drive 88 is maintained in position thereby maintaining first and second jaw members 72, 74 in position grasping tissue therebetween. In this manner, the closure force applied to tissue grasped between jaw members 72, 74 is regulated to maintain a closure force or closure force within a closure force range.

In aspects, the closure force applied to the grasped tissue in the closed position of jaw members 72, 74 may be regulated such that the closure force, measured at a midpoint along the lengths of jaw members 72, 74, may be in a range of (or the jaw force range may be) from about 7.0 lbf to about 11.0 lbf; in other aspects from about 8.0 lbf to about 10.0 lbf; and, in still other aspects, from about 8.5 lbf to about 9.5 lbf.

Continuing with reference to FIGS. 4 and 5, and with additional reference to FIGS. 6 and 7, handle-side pivot and linkage assembly 82 of drive mechanism 80 includes or is coupled to movable handle 34 and has a plurality of linkages 110, 120, 130, 170 and a plurality of pivots 210, 220, 240, 250. Drive-side pivot and linkage assembly 84 is coupled to elongate drive 88 (e.g., via force limiting assembly 85, as detailed above) and likewise has a plurality of linkages 140, 150, 160, 180 and a plurality of pivots 220, 230, 260, 270. Further, handle-side pivot and linkage assembly 82 and drive-side pivot and linkage assembly 84 are coupled to one another such that, as detailed below, actuation of movable handle 34 actuates handle-side pivot and linkage assembly 82 which, in turn, actuates drive-side pivot and linkage assembly 84 which, in turn, actuates elongate drive 88 (directly or indirectly such as, in aspects, via force limiting assembly 85) to move jaw members 72, 74 (FIGS. 1-3) between the spaced apart position (FIG. 2) and the approximated position (FIG. 3).

Drive mechanism 80 and, more specifically, handle-side pivot and linkage assembly 82 and drive-side pivot and linkage assembly 84 of drive mechanism 80, are configured to provide increased mechanical advantage, thus reducing the input force at movable handle 34 required to grasp tissue between jaw members 72, 74 (FIGS. 1-3) with sufficient force, (e.g., within the force ranges detailed above) to facilitate tissue sealing. The increased mechanical advantage of drive mechanism 80 additionally or alternatively enables a greater translation distance of elongate drive 88 relative to a range of motion of movable handle 34 required to actuate movable handle 34 between the unactuated and fully actuated positions. Further, drive mechanism 80 provides the above increased mechanical advantage benefits without requiring an increase in or without significantly increasing the footprint of drive mechanism 80, thus enabling drive mechanism 80 to fit within a compact and ergonomic housing 20 that is readily maneuverable and actuatable by the operator.

Handle-side pivot and linkage assembly 82 and drive-side pivot and linkage assembly 84 may be coupled to one another, e.g., such that actuation of movable handle 34 actuates jaw members 72, 74 (FIGS. 1-3), by a common pivot 220 which, as noted above, functions as part of both handle-side pivot and linkage assembly 82 and drive-side pivot and linkage assembly 84. Additionally or alternatively, handle-side pivot and linkage assembly 82 and drive-side pivot and linkage assembly 84 may be coupled to one another by first and second angled linkages 130, 140 of handle-side pivot and linkage assembly 82 and drive-side pivot and linkage assembly 84, respectively, wherein first and second angled linkages 130, 140 are fixed relative to one another to define a fixed angle therebetween (e.g., wherein linkages 130, 140 are defined by portions of an integral body 89 or are defined by other components fixed relative to one another). Further, and as also described in greater detail below, handle-side pivot and linkage assembly 82 and/or drive-side pivot and linkage assembly 84 may be configured as four-bar mechanical linkage assemblies.

Referring still to FIGS. 4-7, linkages 110-160 are movable relative to housing 20 (and, thus, are referred to as “movable linkages”) and linkages 170, 180 are fixed relative to housing 20 (and, thus, are referred to as “fixed linkages”). Movable linkages 110-160 include: a handle linkage 110 defined by at least a portion of movable handle 34 and/or one or more components fixed relative to movable handle 34; a handle-side intermediate linkage 120; first and second angled linkages 130, 140 defining a fixed angle therebetween (e.g., wherein linkages 130, 140 are defined by portions of an integral body 89 or are defined by other components fixed relative to one another); a drive-side intermediate linkage 150; and a drive linkage 160. Fixed linkages 170, 180 may be at least partially defined by portions of housing 20, e.g., the portion of housing 20 extending between pivots 210 and 220 and the portion of housing 20 extending between pivots 220 and 230, respectively, and/or other components fixed relative to housing 20 such that fixed linkages 170, 180 are fixed relative to housing 20.

Pivots 210-230 are disposed in fixed position (but rotatable) relative to housing 20 (and, thus, are referred to as “fixed pivots”) and pivots 240-270 are movable (and rotatable) relative to housing 20 (and, thus, are referred to as “floating pivots”). Fixed pivots 210-230 include: a handle pivot 210 pivotably coupling movable handle 34 to housing 20; a common pivot 220 pivotably coupling first and second angled linkages 130, 140 to housing 20; and a drive pivot 230 pivotably coupling drive linkage 160 to housing 20. Floating pivots 240-270 include: a handle-side floating pivot 240 pivotably coupling handle-side intermediate linkage 120 with handle linkage 110; first and second relatively fixed pivots 250, 260 (e.g., fixed relative to integral body 89) pivotably coupling first and second angled linkages 130, 140 to handle-side intermediate linkage 120 and drive-side intermediate linkage 150, respectively; and a drive-side floating pivot 270 pivotably coupling drive-side intermediate linkage 150 with drive linkage 160.

Handle pivot 210, as noted above, pivotably couples movable handle 34 to housing 20, thus enabling pivoting of movable handle 34 relative to housing 20 and about handle pivot 210 from the unactuated position (e.g., wherein movable handle 34 is farther from fixed handle 32) towards the actuated position (e.g., wherein movable handle 34 is closer to fixed handle 32). A grasping portion 35 of movable handle 34 (e.g., the portion 35 of movable handle 34 that is external of housing 20 to enable grasping and manipulation by the operator) is movable in a first direction from the unactuated position towards the actuated position and in a second, opposite direction from the actuated position back towards the unactuated position. The first direction may be proximally towards fixed handle 32 and, thus, the second direction may be distally away from fixed handle 32.

Handle-side floating pivot 240, as noted above, pivotably couples handle-side intermediate linkage 120 with handle linkage 110. Handle pivot 210 and handle-side floating pivot 240 are spaced apart from one another along movable handle 34, thus defining handle linkage 110 therebetween. Further, grasping portion 35 of movable handle 34 and handle-side floating pivot 240 are disposed along movable handle 34 on the same side as one another relative to handle pivot 210. That is, handle-side floating pivot 240 is disposed between handle pivot 210 and grasping portion 35, although it is also contemplated that, in aspects, grasping portion 35 is disposed between handle pivot 210 and handle-side floating pivot 240. As a result of this configuration, actuation of movable handle 34 (handle linkage 110) in the first direction, e.g., proximally, likewise urges handle-side intermediate linkage 120 in the first direction, e.g., proximally. Similarly, movement of movable handle 34 (handle linkage 110) in the second direction, e.g., distally, urges handle-side intermediate linkage 120 in the second direction, e.g., distally. In aspects, a biasing member (not shown), e.g., a spring, is provided to bias movable handle 34 towards the unactuated position, corresponding to the spaced apart position of jaw members 72, 74 (see FIG. 2).

Continuing with reference to FIGS. 4-7, first relatively fixed pivot 250 pivotably couples handle-side intermediate linkage 120 and first angled linkage 130 with one another. First relatively fixed pivot 250 is a floating pivot relative to housing 20 and is pivotably coupled to handle-side intermediate linkage 120 at a position spaced-apart from handle-side floating pivot 240. For example, in aspects, handle-side floating pivot 240 is disposed at a distal end portion of handle-side intermediate linkage 120 while first relatively fixed pivot 250 is disposed at a proximal end portion of handle-side intermediate linkage 120. However, other configurations are also contemplated.

Second relatively fixed pivot 260 is a floating pivot relative to housing 20 and pivotably couples second angled linkage 140 with drive-side intermediate linkage 150. As noted above, first and second relatively fixed pivots 250, 260 are disposed in fixed position relative to one another (and in fixed position relative to common pivot 220) and first and second angled linkages 130, 140 are disposed in fixed position relative to one another thus defining a fixed angle therebetween. The fixed angle defined between first and second angled linkages 130, 140 may be, in aspects, approximately 90 degrees (such that first and second angled linkages 130, 140 are substantially perpendicular relative to one another), although other configurations are also contemplated such as, for example, wherein the fixed angle defined between first and second angled linkage 130, 140 is from about 45 degrees to about 135 degrees.

First and second relatively fixed pivots 250, 260 are spaced apart relative to one another a fixed distance. First and second relatively fixed pivots 250, 260 are also spaced apart from common pivot 220 fixed distances. In aspects, the fixed distances between each of first and second relatively fixed pivots 250, 260 and common pivot 220 are substantially equal to one another such that first and second angled linkages 130, 140 define substantially equal lengths. In aspects, as noted above, an integral body 89 (e.g., a single monolithic body or plural components fixed relative to one another to define a body) is pivotably coupled to housing 20 about common pivot 220, defines first and second angled linkages 130, 140, and is coupled to handle-side and drive-side intermediate linkages 120, 150 via first and second relatively fixed pivots 250, 260, respectively. Whether fixed by integral body 89 or in any other suitable manner, first and second angled linkages 130, 140 are rotatable together relative to housing 20 about common pivot 220. In aspects where first and second angled linkages 130, 140 define substantially equal lengths, first and second relatively fixed pivots 250, 260 share a circular rotation path as first and second angled linkages 130, 140 are rotated about common pivot 220. First relatively fixed pivot 250 is positioned relative to common pivot 220 and drive-side intermediate linkage 150 such that actuation of movable handle 34 proximally urges first relatively fixed pivot 250 to rotate about common pivot 220 in a counterclockwise direction (based on the orientation shown in FIGS. 4-7).

Drive-side floating pivot 270, as noted above, pivotably couples drive-side intermediate linkage 150 and drive linkage 160 with one another. Drive-side floating pivot 270, more specifically, is pivotably coupled to drive-side intermediate linkage 150 at a position spaced-apart from second relatively fixed pivot 260 along drive-side intermediate linkage 150. For example, in aspects, drive-side floating pivot 270 is disposed at a distal end portion of drive-side intermediate linkage 150 while second relatively fixed pivot 260 is disposed at a proximal end portion of drive-side intermediate linkage 150. However, other configurations are also contemplated.

Drive linkage 160 defines a first end portion 162, a second, opposite end portion 164, and a drive portion 166 disposed between first and second end portions 162, 164, respectively. Drive portion 166 may be equally spaced between first and second end portions 162, 164 or may be disposed closed to first end portion 162 or second end portion 164. Further, drive portion 166 is disposed within the receiving area of mandrel 87a, e.g., between the proximal and distal rims of mandrel 87a, such that movement of drive linkage 160 in a proximal direction urges drive portion 166 into the proximal rim of mandrel 87a to urge mandrel 87a proximally and such that movement of drive linkage 160 in a distal direction urges drive portion 166 into the distal rim of mandrel 87a to urge mandrel 87a distally. Drive portion 166 may define arcuate protruding proximal and distal surfaces to facilitate rotational sliding contact with the proximal and distal rims of mandrel 87a, respectively, thus facilitating respective proximal and distal urging of mandrel 87a in response to movement of drive linkage 160.

Drive pivot 230 is fixed relative to housing 20 and pivotably couples drive linkage 160 to housing 20. Drive pivot 230 is spaced apart relative to drive-side floating pivot 270 along drive linkage 160. For example, drive-side floating pivot 270 may be disposed at first end portion 162 of drive linkage 160 while drive pivot 230 is disposed at second end portion 164 of drive linkage 160. Further, drive portion 166 and, thus, the coupling of drive linkage 160 with mandrel 87a, is disposed between drive pivot 230 and drive-side floating pivot 270. Thus, in response to drive-side intermediate linkage 150 urging (e.g., pulling) first end 162 of drive linkage 160 in a first direction, e.g., proximally, drive linkage 160 is pivoted about drive pivot 230 with drive portion 166 likewise moving proximally. On the other hand, in response to drive-side intermediate linkage 150 urging (e.g., pushing) first end 162 of drive linkage 160 in a second, opposite direction, e.g., distally, drive linkage 160 is pivoted about drive pivot 230 with drive portion 166 likewise moving distally.

In aspects, drive-side floating pivot 270 is guided along and/or configured to movement along an arcuate path relative to housing 20 via receipt of either or both free ends of drive-side floating pivot 270 within an arcuate track 29 (FIGS. 4 and 5) disposed on either or both internal surfaces of housing 20 (or other component fixed relative to housing 20).

Still referring to FIGS. 4-7, as noted above, handle-side pivot and linkage assembly 82 and/or drive-side pivot and linkage assembly 84 may be configured as four-bar mechanical linkage assemblies. More specifically, the four-bar mechanical linkage assembly of handle-side pivot and linkage assembly 82 may be defined by linkages 110, 120, 130 functioning as the three movable linkages of the four-bar mechanical linkage assembly and with linkage 170, e.g., the portion of housing 20 (and/or other components fixed relative to housing 20) extending between pivots 210 and 220, functioning as the fixed linkage of the four-bar mechanical linkage assembly of handle-side pivot and linkage assembly 82. The fixed pivots of the four-bar mechanical linkage assembly of handle-side pivot and linkage assembly 82 are pivots 210, 220 and the floating pivots of the four-bar mechanical linkage assembly of handle-side pivot and linkage assembly 82 are pivots 240, 250. The four-bar mechanical linkage assembly of drive-side pivot and linkage assembly 84 may be defined by linkages 140, 150, 160 functioning as the three movable linkages of the four-bar mechanical linkage assembly and with linkage 180, e.g., the portion of housing 20 (and/or other components fixed relative to housing 20) extending between pivots 220 and 230, functioning as the fixed linkage of the four-bar mechanical linkage assembly of handle-side pivot and linkage assembly 84. The fixed pivots of the four-bar mechanical linkage assembly of drive-side pivot and linkage assembly 84 are pivots 220, 230 and the floating pivots of the four-bar mechanical linkage assembly of drive-side pivot and linkage assembly 84 are pivots 260, 270.

In aspects, linkages 110, 120, 130, 140, 150, and 170 of drive assembly 80 as well as pivots 210, 220, 240, 250, 260, 270 of drive assembly 80 are entirely disposed on a first side of a longitudinal axis “X-X” defined by elongate drive 88 (e.g., above the longitudinal axis “X-X” based on the orientation illustrated in FIGS. 4 and 5), while linkages 160, 180 intersect longitudinal axis “X-X” with portions disposed on the first and second, opposite sides of longitudinal axis “X-X,” and with pivot 230 is disposed on the second side of longitudinal axis “X-X” (e.g., below the longitudinal axis “X-X” based on the orientation illustrated in FIGS. 4 and 5). However, other configurations are also contemplated.

Any of the linkages 110-180 of drive assembly 80 may be configured as single links or bifurcated links such as, for example, to enable another link to pass between the link portions of the bifurcated link. Additionally or alternatively, any of the linkages 110-180 may be substantially linear, may be curved or include curved portions, may include angled segments, combinations thereof, etc. Other suitable configurations of linkages 110-180 are also contemplated. Further, any of the pivots 210-270 of drive assembly 80 may be formed by pivot pins, pivot bosses, etc. and may be continuous pivots or split pivots (e.g., pivots including first and second pivot portions defining a space or other structure therebetween). Other suitable configurations of pivots 210-270 are also contemplated.

In use, upon actuation of movable handle 34 proximally from the unactuated position (FIGS. 4 and 6) towards the actuated position (FIGS. 5 and 7), handle linkage 110 is pivoted proximally about handle pivot 210, thereby pushing handle-side intermediate linkage 120 proximally. Handle-side intermediate linkage 120 is also pivoted relative to handle linkage 110 about handle-side floating pivot 240 to enable this movement. This movement of handle-side intermediate linkage 120, in turn, urges first and second angled linkages 130, 140 (and, in aspects where provided, integral body 89) to pivot relative to housing 20 about fixed common pivot 220, e.g., in a counterclockwise direction based on the orientation illustrated in FIGS. 4-7. Relative pivoting between handle-side intermediate linkage 120 and first and second angled linkages 130, 140 is also affected to enable this movement. This pivoting of first and second angled linkages 130, 140, in turn, pulls drive-side intermediate linkage 150 proximally and also pivots drive-side intermediate linkage 150 relative to first and second angled linkages 130, 140 about second relatively fixed pivot 260 to facilitate the proximal pulling of drive-side intermediate linkage 150. This proximal pulling of drive-side intermediate linkage 150, in turn, pulls first end portion 162 of drive linkage 160 proximally, thereby urging drive linkage 160 to rotate relative to housing 20 about drive pivot 230, e.g., in a clockwise direction based on the orientation illustrated in FIGS. 4-7. As detailed above, proximal movement of drive linkage 160 urges drive portion 166 of drive linkage 160 proximally to urge mandrel 87a into compression spring 87c. If the force applied by first and second jaw members 72, 74 (FIGS. 1-3) to tissue disposed therebetween is less than the threshold force, this urging of mandrel 87a into compression spring 87c translates compression spring 87c proximally, thereby translating elongate drive 88 to pivot first and second jaw members 72, 74 (FIGS. 1-3) from the spaced apart position (FIG. 2) towards the approximated position (FIG. 3). If the force applied by first and second jaw members 72, 74 (FIGS. 1-3) to tissue disposed therebetween meets the threshold force, the urging of mandrel 87a into compression spring 87c compresses compression spring 87c to maintain elongate drive 88 in position and, thus, maintain the position of first and second jaw members 72, 74 (FIGS. 1-3) and the closure force applied thereby.

Upon release or return of movable handle 34 distally from the actuated position (FIGS. 5 and 7) back towards the unactuated position (FIGS. 4 and 6), handle linkage 110 is pivoted distally about handle pivot 210, thereby pulling handle-side intermediate linkage 120 distally to urge first and second angled linkages 130, 140 to pivot relative to housing 20 about fixed common pivot 220, e.g., in a clockwise direction based on the orientation illustrated in FIGS. 4-7, to thereby push drive-side intermediate linkage 150 distally and pivot drive linkage 160 distally, thereby pulling mandrel 87a, compression spring 87c, and elongate drive 88 distally to pivot first and second jaw members 72, 74 (FIGS. 1-3) from the approximated position (FIG. 3) back towards the spaced apart position (FIG. 2).

As an alternative to the above-detailed configuration wherein actuation of movable handle 34 proximally from the unactuated position (FIGS. 4 and 6) towards the actuated position (FIGS. 5 and 7) moves mandrel 87a (and, thus, elongate drive 88) proximally (e.g., in the same direction), drive mechanism 80 may be configured such that proximal actuation of movable handle 34 moves mandrel 87a (and, thus, elongate drive 88) distally (e.g., in the opposite direction). This may be accomplished, for example and without limitation, by moving the location of pivot 250 relative to pivot 220 (e.g., to an opposing position) such that first and second angled linkages 130, 140 (and, in aspects where provided, integral body 89) are pivoted relative to housing 20 about fixed common pivot 220 in a clockwise direction based on the orientation illustrated in FIGS. 4-7 in response to proximal actuation of movable handle 34. In such a configuration, pivoting of first and second angled linkages 130, 140 in the clockwise direction urges drive-side intermediate linkage 150 distally to thereby pivot drive linkage 160 distally and, in turn, urge mandrel 87a (and, thus, elongate drive 88) distally. In such configurations, washer 87b may be positioned distally of mandrel 87a with compression spring 87c disposed therebetween to enable the same force-limiting effect as detailed above. Other suitable linkage(s) and/or pivot(s) location changes are also contemplated to achieve the above-detailed urging of mandrel 87a in an opposite direction relative to the actuation direction of movable handle 34.

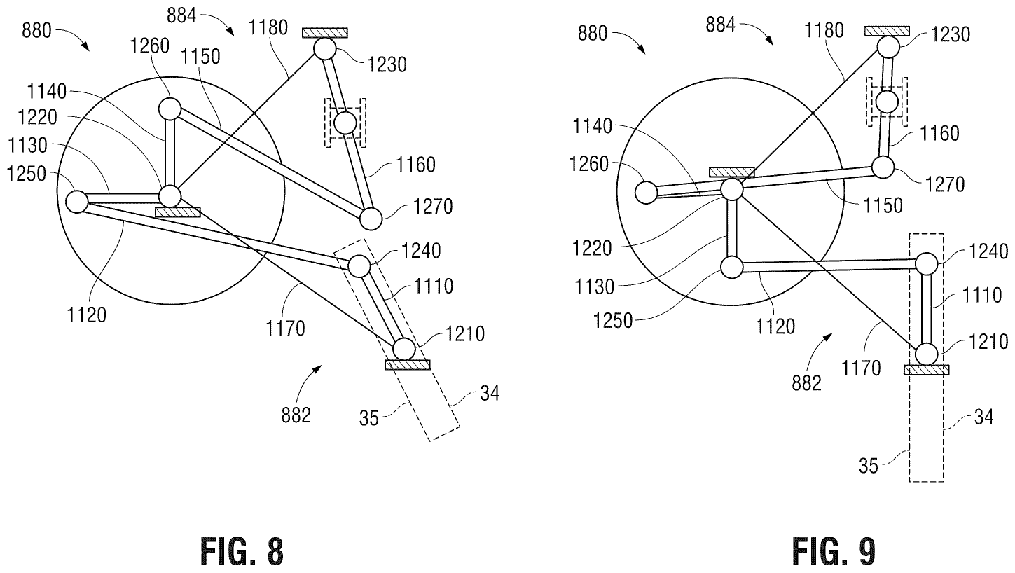

Turning to FIGS. 8 and 9, another drive mechanism 880 provided in accordance with the present disclosure and configured for use with electrosurgical forceps 10 (FIG. 1) or any other suitable surgical instrument is shown. Drive mechanism 880 is similar to drive mechanism 80 (FIGS. 4-7) and may include any of the aspects and features of drive mechanism 80 (FIGS. 4-7). Thus, only differences between drive mechanism 880 and drive mechanism 80 (FIGS. 4-7) are described in detail below while similarities are summarily described or omitted entirely.

Drive mechanism 880 includes a handle-side pivot and linkage assembly 882 and a drive-side pivot and linkage assembly 884. Handle-side pivot and linkage assembly 882 includes or is coupled to movable handle 34 and has a plurality of linkages 1110, 1120, 1130, 1170 and a plurality of pivots 1210, 1220, 1240, 1250. Drive-side pivot and linkage assembly 884 is coupled to elongate drive 88 (FIGS. 4 and 5) and likewise has a plurality of linkages 1140, 1150, 1160, 1180 and a plurality of pivots 1220, 1230, 1260, 1270. Handle-side pivot and linkage assembly 882 and drive-side pivot and linkage assembly 884 are coupled to one another and either or both may define four-bar mechanical linkage assemblies.

Linkages 1110-1160 are movable linkages and linkages 1170, 1180 are fixed linkages. Further, pivots 1210-1230 are fixed pivots and pivots 1240-1270 are movable pivots. Drive mechanism 880 differs from drive mechanism 80 (FIGS. 4-7) in that grasping portion 35 of movable handle 34 and handle-side floating pivot 1240 are disposed along movable handle 34 on opposite sides of handle pivot 1210. As a result of this configuration, actuation of movable handle 34 (handle linkage 1110) in the first direction, e.g., proximally, urges handle-side intermediate linkage 1120 in a second, opposite direction, e.g., distally. The angles and/or lengths of some or all of linkages 1110-1180 and the relative locations of some or all of pivots 1210-1270 of drive mechanism 880 may also vary from those of drive mechanism 80 (FIGS. 4-7) to account for the above difference. In aspects, handle pivot 1210 may be disposed above the longitudinal axis “X-X” (FIGS. 4 and 5), although handle pivot 1210 may alternatively be aligned on or disposed below longitudinal axis “X-X” (FIGS. 4 and 5).
Aspects of this disclosure may be further described by reference to the following numbered paragraphs:
1. A surgical instrument, comprising: a housing; an end effector assembly distally spaced from the housing, the end effector assembly including first and second jaw members, at least one of the first or second jaw members movable relative to the other between a spaced apart position and an approximated position; an elongate drive extending from the housing to the end effector assembly, the elongate drive coupled to the at least one of the first or second jaw members such that translation of the elongate drive moves the at least one of the first or second jaw members relative to the other; a movable handle coupled to the housing and movable relative to the housing between an un-actuated position and an actuated position; and a drive mechanism at least partially disposed within the housing, the drive mechanism including: a first four-bar mechanical linkage assembly, wherein the movable handle defines or is coupled to a handle linkage of the first four-bar mechanical linkage assembly; and a second four-bar mechanical linkage assembly, wherein a drive linkage of the second four-bar mechanical linkage assembly is coupled to the elongate drive such that movement of the drive linkage of the second four-bar mechanical linkage assembly translates the elongate drive, wherein the first and second four-bar mechanical linkage assemblies are coupled to one another, thereby coupling the movable handle with the elongate drive such that movement of the movable handle from the un-actuated position towards the actuated position translates the elongate drive to thereby move the at least one of the first or second jaw members from the spaced-apart position towards the approximated position.
2. The surgical instrument according to paragraph 1, wherein the first and second four-bar mechanical linkage assemblies are configured such that movement of the movable handle in a rotational direction drives rotation of the drive linkage of the second four-bar mechanical linkage assembly in the same rotational direction.
3. The surgical instrument according to paragraph 1 or 2, wherein the first and second four-bar mechanical linkage assemblies are configured such that movement of the movable handle in a direction translates the elongate drive in the same direction.
4. The surgical instrument according to any preceding paragraph, wherein the first and second four-bar mechanical linkage assemblies share a common pivot, the common pivot coupling the first and second four-bar mechanical linkage assemblies with one another.
5. The surgical instrument according to paragraph 4, wherein the common pivot is fixed relative to the housing.
6. The surgical instrument according to paragraph 4 or 5, wherein a linkage of each of the first and second four-bar mechanical linkage assemblies is pivotably coupled to the common pivot.
7. The surgical instrument according to paragraph 6, wherein the linkages of the first and second four-bar mechanical linkage assemblies are disposed in fixed orientation relative to one another.
8. The surgical instrument according to any preceding paragraph, wherein the first four-bar mechanical linkage assembly includes a fixed linkage and three movable linkages, and wherein the handle linkage is one of the three movable linkages.
9. The surgical instrument according to paragraph 8, wherein the housing defines the fixed linkage of the first four-bar mechanical linkage assembly.
10. The surgical instrument according to any preceding paragraph, wherein the second four-bar mechanical linkage assembly includes a fixed linkage and three movable linkages, and wherein the drive linkage is one of the three movable linkages of the second four-bar mechanical linkage assembly.
11. The surgical instrument according to paragraph 10, wherein the housing defines the fixed linkage of the second four-bar mechanical linkage assembly.
12. The surgical instrument according to any preceding paragraph, wherein the movable handle is pivotably coupled to the housing about a fixed pivot of the first four-bar mechanical linkage assembly.
13. The surgical instrument according to any preceding paragraph, wherein the drive linkage is pivotably coupled to the housing about a fixed pivot of the second four-bar mechanical linkage assembly.
14. The surgical instrument according to any preceding paragraph, wherein the drive mechanism further includes a mandrel coupled to the elongate drive, wherein the drive linkage is coupled to the mandrel such that movement of the drive linkage of the second four-bar mechanical linkage assembly translates mandrel to thereby translate the elongate drive.
15. The surgical instrument according to paragraph 14, wherein the mandrel is coupled to the elongate drive by a compression spring of the drive mechanism such that translation of the mandrel applies force to the compression spring which, in turn, applies force to the elongate drive.
16. The surgical instrument according to any preceding paragraph, wherein the movable handle defines the handle linkage of the first four-bar mechanical linkage assembly.
17. A surgical instrument, comprising: a housing; an end effector assembly distally spaced from the housing, the end effector assembly including first and second jaw members, at least one of the first or second jaw members movable relative to the other between a spaced apart position and an approximated position for grasping tissue therebetween; an elongate drive extending from the housing to the end effector assembly, the elongate drive coupled to the at least one of the first or second jaw members such that translation of the elongate drive moves the at least one of the first or second jaw members relative to the other; a movable handle coupled to the housing and movable relative to the housing between an un-actuated position and an actuated position; and a drive mechanism at least partially disposed within the housing, the drive mechanism including a plurality of movable linkages coupled between the movable handle and the elongate drive such that movement of the movable handle from the un-actuated position towards the actuated position translates the elongate drive to thereby move the at least one of the first or second jaw members from the spaced-apart position towards the approximated position, the plurality of movable linkages including: a handle linkage pivotably coupled to the housing about a first fixed pivot; a drive linkage pivotably coupled to the housing about a second fixed pivot; a first intermediate linkage coupled to the handle linkage; a second intermediate linkage coupled to the drive linkage; and first and second angled linkages coupled between the first and second intermediate linkages, the first and second angled linkages pivotably coupled to the housing about a third fixed pivot and defining a fixed angle therebetween.
18. The surgical instrument according to paragraph 17, wherein the plurality of movable linkages is configured such that movement of the movable handle in a rotational direction drives rotation of the drive linkage in the same rotational direction.
19. The surgical instrument according to paragraph 17 or 18, wherein the plurality of movable linkages is configured such that movement of the movable handle in a direction translates the elongate drive in the same direction.
20. The surgical instrument according to any one of paragraphs 17-19, wherein at least one of: the handle linkage, the first intermediate linkage, the first angled linkage, and the housing define a first four-bar mechanical linkage assembly; or the drive linkage, the second intermediate linkage, the second angled linkage, and the housing define a second four-bar mechanical linkage assembly.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A surgical instrument, comprising:
a housing;
an end effector assembly distally spaced from the housing, the end effector assembly including first and second jaw members, at least one of the first or second jaw members movable relative to the other between a spaced apart position and an approximated position;
an elongate drive extending from the housing to the end effector assembly, the elongate drive coupled to the at least one of the first or second jaw members such that translation of the elongate drive moves the at least one of the first or second jaw members relative to the other;
a movable handle coupled to the housing and movable relative to the housing between an un-actuated position and an actuated position; and
a drive mechanism at least partially disposed within the housing, the drive mechanism including:
a first four-bar mechanical linkage assembly, wherein the movable handle defines or is coupled to a handle linkage of the first four-bar mechanical linkage assembly; and
a second four-bar mechanical linkage assembly, wherein a drive linkage of the second four-bar mechanical linkage assembly is coupled to the elongate drive such that movement of the drive linkage of the second four-bar mechanical linkage assembly translates the elongate drive,
wherein the first and second four-bar mechanical linkage assemblies are coupled to one another, thereby coupling the movable handle with the elongate drive such that movement of the movable handle from the un-actuated position towards the actuated position translates the elongate drive to thereby move the at least one of the first or second jaw members from the spaced-apart position towards the approximated position.

2. The surgical instrument of claim 1, wherein the first and second four-bar mechanical linkage assemblies are configured such that movement of the movable handle in a rotational direction drives rotation of the drive linkage of the second four-bar mechanical linkage assembly in the same rotational direction.

3. The surgical instrument of claim 1, wherein the first and second four-bar mechanical linkage assemblies are configured such that movement of the movable handle in a direction translates the elongate drive in the same direction.

4. The surgical instrument of claim 1, wherein the first and second four-bar mechanical linkage assemblies share a common pivot, the common pivot coupling the first and second four-bar mechanical linkage assemblies with one another.

5. The surgical instrument of claim 4, wherein the common pivot is fixed relative to the housing.

6. The surgical instrument of claim 4, wherein a linkage of each of the first and second four-bar mechanical linkage assemblies is pivotably coupled to the common pivot.

7. The surgical instrument of claim 6, wherein the linkages of the first and second four-bar mechanical linkage assemblies are disposed in fixed orientation relative to one another.

8. The surgical instrument of claim 1, wherein the first four-bar mechanical linkage assembly includes a fixed linkage and three movable linkages, and wherein the handle linkage is one of the three movable linkages.

9. The surgical instrument of claim 8, wherein the housing defines the fixed linkage of the first four-bar mechanical linkage assembly.

10. The surgical instrument of claim 1, wherein the second four-bar mechanical linkage assembly includes a fixed linkage and three movable linkages, and wherein the drive linkage is one of the three movable linkages of the second four-bar mechanical linkage assembly.

11. The surgical instrument of claim 10, wherein the housing defines the fixed linkage of the second four-bar mechanical linkage assembly.

12. The surgical instrument of claim 1, wherein the movable handle is pivotably coupled to the housing about a fixed pivot of the first four-bar mechanical linkage assembly.

13. The surgical instrument of claim 1, wherein the drive linkage is pivotably coupled to the housing about a fixed pivot of the second four-bar mechanical linkage assembly.

14. The surgical instrument of claim 1, wherein the drive mechanism further includes a mandrel coupled to the elongate drive, wherein the drive linkage is coupled to the mandrel such that movement of the drive linkage of the second four-bar mechanical linkage assembly translates mandrel to thereby translate the elongate drive.

15. The surgical instrument of claim 14, wherein the mandrel is coupled to the elongate drive by a compression spring of the drive mechanism such that translation of the mandrel applies force to the compression spring which, in turn, applies force to the elongate drive.

16. The surgical instrument of claim 1, wherein the movable handle defines the handle linkage of the first four-bar mechanical linkage assembly.

17. A surgical instrument, comprising:
a housing;
an end effector assembly distally spaced from the housing, the end effector assembly including first and second jaw members, at least one of the first or second jaw members movable relative to the other between a spaced apart position and an approximated position for grasping tissue therebetween;
an elongate drive extending from the housing to the end effector assembly, the elongate drive coupled to the at least one of the first or second jaw members such that translation of the elongate drive moves the at least one of the first or second jaw members relative to the other;
a movable handle coupled to the housing and movable relative to the housing between an un-actuated position and an actuated position; and
a drive mechanism at least partially disposed within the housing, the drive mechanism including a plurality of movable linkages coupled between the movable handle and the elongate drive such that movement of the movable handle from the un-actuated position towards the actuated position translates the elongate drive to thereby move the at least one of the first or second jaw members from the spaced-apart position towards the approximated position, the plurality of movable linkages including:
a handle linkage pivotably coupled to the housing about a first fixed pivot;
a drive linkage pivotably coupled to the housing about a second fixed pivot;
a first intermediate linkage coupled to the handle linkage;
a second intermediate linkage coupled to the drive linkage; and
first and second angled linkages coupled between the first and second intermediate linkages, the first and second angled linkages pivotably coupled to the housing about a third fixed pivot and defining a fixed angle therebetween.

18. The surgical instrument of claim 17, wherein the plurality of movable linkages is configured such that movement of the movable handle in a rotational direction drives rotation of the drive linkage in the same rotational direction.

19. The surgical instrument of claim 17, wherein the plurality of movable linkages is configured such that movement of the movable handle in a direction translates the elongate drive in the same direction.

20. The surgical instrument of claim 17, wherein at least one of:
the handle linkage, the first intermediate linkage, the first angled linkage, and the housing define a first four-bar mechanical linkage assembly; or
the drive linkage, the second intermediate linkage, the second angled linkage, and the housing define a second four-bar mechanical linkage assembly.
